(19) United States
(12) Patent Application Publication
(10) Pub. No.: US 2010/0120050 A1
(43) Pub. Date: May 13, 2010

(54) Biomarkers For Assessing Altherosclerotic Potential

(75) Inventors: Kapil Gadkar, Mountain View, CA (US); Ananth Kadambi, San Mateo, CA (US); Cecelia Pearson, Redwood City, CA (US); Lynn Powell, San Mateo, CA (US); Scott Siler, Castro Valley, CA (US); Jeff Trimmer, San Carlos, CA (US)

Correspondence Address: ENTELOS, INC.;c/o Law Offices of Karen E. Flick, P.O. Box 515, El Granada, CA, 94018-0515, US

(73) Assignee: ENTELOS, INC., Foster City, CA (US)

(21) Appl. No.: 12/616,701

(22) Filed: Nov. 11, 2009

Related U.S. Application Data
(60) Provisional application No. 61/113,417, filed on Nov. 11, 2008.

(51) Int. Cl. C12Q 1/68

(52) U.S. Cl. 435/6

(57) ABSTRACT

The invention also provides methods, apparatuses and reagents useful for predicting future atherosclerosis based on expression levels of genes selected from the set of 68 genes with differential expression in response to pioglitazone and rosiglitazone. The invention also discloses reagent sets and biomarkers for predicting progression of atherosclerosis induced by anti-diabetic therapy in a subject. In one particular embodiment the invention provides a method for predict whether a compound will induce atherosclerosis using gene expression data from sub-acute treatments.

### CROSS-REFERENCE TO RELATED APPLICATIONS

This application claims the benefit of U.S. provisional patent application No. 61/113,417, filed 11 Nov. 2008, incorporated herein by reference in its entirety.

### TECHNICAL FIELD

This invention provides a novel means of discriminating between therapeutic compounds having a deleterious, pro-atherosclerotic effect on lipoprotein particle number and distribution, and those compounds having anti-atherosclerotic, protective effect.

### BACKGROUND OF THE INVENTION

Obesity and diabetes are independent risk factors for cardiovascular events, likely due to an acceleration of atherosclerosis progression. Both diseases are characterized by changes in serum levels of lipoprotein particles resulting in the so-called atherogenic lipid triad (low HDL-cholesterol, raised triglycerides, and a preponderance of small, dense LDL particles). Development of therapeutics for these metabolic disorders is typically focused on treating the symptoms of elevated bodyweight, fasting and post-prandial blood glucose, impaired insulin sensitivity in muscle, liver and adipose tissue, and impaired pancreatic function. Animal models of atherosclerosis do not accurately represent the human physiology of lipoprotein metabolism and plaque growth and development, moreover they are not typically used when pre-clinically evaluating prospective therapeutic candidates for diabetes. This has led to a situation where during clinical trials and post-marketing, therapeutic interventions for obesity and diabetes result in little observed effect on plaque endpoints (rimonabant, marketed in Europe as Acomplia) or paradoxically increased risk of cardiovascular events (rosiglitazone, marketed as AVANDIA®).

The alpha, gamma and delta or beta subtypes of peroxisome proliferator activated receptors (PPAR), which are nuclear hormone receptors, are targets for controlling lipid, glucose and energy homeostasis. Highly potent PPARγ agonists, PPARα/γ dual agonists, PPARpan agonists, and alternative PPAR ligands such as partial agonists or selective PPAR modulators (SPPARMs) are being pursued as therapeutics designed to improve insulin sensitivity. A recent meta-analysis of clinical trial data showed that the PPARγ agonist AVANDIA® (rosiglitazone maletate) was associated with increased CV events (Nissen and Wolski, “Effect of rosiglitazone on the risk of myocardial infarction and death from cardiovascular causes.” N Engl J. Med. 2007 356(24):2457-71), while a structurally related PPARγ agonist, Actos® (pioglitazone HCl), was associated with reduced CV events (Lincoff, et al. “Pioglitizone and risk of cardiovascular events in patients with type 2 diabetes mellitus: a meta-analysis of randomized trials.” JAMA 2007 298(10):1180-8), despite a similar effect on diabetes endpoints for both drugs.

Thus a need exists for methods of identifying which compounds used for treating metabolic disorders have increased risk for cardiovascular events. A need also exists for identifying those compounds that can decrease cardiovascular risk, in addition to having efficacy against metabolic disorders.

### SUMMARY OF THE INVENTION

One aspect of the present invention provides methods of predicting adverse effects on cardiovascular risk resulting from therapeutics that produce changes in patient lipoprotein particle numbers and distributions.

One aspect of the invention provides biomarkers for assessing atherosclerotic potential of an anti-diabetic therapy in a subject, said biomarker comprising a measurement of expression of each of a plurality of genes selected from those listed in Table 2. Preferably the plurality of genes comprises at least three, at least five or at least eight genes selected from Table 2. Preferably, the plurality of genes includes at least one of malic enzyme 1 (accession No. M30596), perilipin (Accession No. AI406700), pyruvate carboxylase (Accession No. BG376902), acetyl-Coenzyme A acyltransferase 2 (mitochondrial 3-oxoacyl-Coenzyme Athiolase)) Accession No. BI282488), 3-hydroxy-3-methylglutaryl-Coenzyme A reductase (accession No. BM390399), and apolipoprotein E (Accession No. J02582).

Another aspect of the invention provides methods for testing whether a compound will induce atherosclerosis in a test subject, the method comprising: administering a dose of the compound to at least one test subject; after a selected time period, obtaining a biological sample from the at least one test subject; measuring the expression levels in the biological sample of at least a plurality of genes selected from those listed in Table 2; and determining whether the sample is in the positive class for induction of atherosclerosis using a biomarker comprising at least the plurality of genes for which the expression levels are measured. The plurality of genes, preferably, comprises at least three, at least five or at least eight genes selected from those listed in Table 2, below. In one implementation, the biological sample comprises liver tissue. In another implementation of the method, the expression levels are measured as log₁₀ ratios of compound-treated biological sample to a compound-untreated biological sample. In certain implementations, the selected period of time is equal to or less than about 7 days, more preferably equal to or less than about three days and most preferably equal to or less than about one day. In certain implementations, the selected period of time can be as short as three hours, one hour or even thirty minutes.

Another aspect of the invention provides reagent sets comprising a plurality of polynucleotides or polypeptides capable of assessing the amount of expression of a plurality of genes selected from those listed in Table 2. In certain implementations, the plurality of genes includes at least 3 genes, more preferably at least 5 genes and ever more preferably at least 8 genes, selected from those listed in Table 2. In another implementation, the reagent set consists essentially of polynucleotides or polypeptides capable of assessing the amount of expression of genes selected from Table 2.

It will be appreciated by one of skill in the art that the embodiments summarized above may be used together in any suitable combination to generate additional embodiments not expressly recited above, and that such embodiments are considered to be part of the present invention.

### BRIEF DESCRIPTION OF THE DRAWINGS

FIG. 1 illustrates predicted percent atheroma volume (PAV) changes over 5 years for virtual patients with profiles reflecting treatment with rosiglitazone (solid squares) or pioglitazone (open squares).

FIG. 2 illustrates predicted changes in plaque stability over 5 years for virtual patients with profiles reflecting treatment with rosiglitazone (solid squares) or pioglitazone (open squares).

### DETAILED DESCRIPTION OF THE INVENTION

Clinical data suggest that rosiglitazone causes an increase in circulating LDL particles and a decrease in HDL particles, while pioglitazone has the opposite effect. An in silico, mechanistic model of human cardiovascular disease, the Cardiovascular PhysioLab® platform (described in greater detail in patent application publication 2008-0249751 A1, incorporated herein by reference in its entirety) was used to test the hypothesis that these differences underlie the opposite effects of rosiglitazone and pioglitazone on CV event rates. Plaque progression over five years was simulated in virtual patients with baseline lipoprotein profiles representative of patients treated with rosiglitazone and pioglitazone. Simulations predicted that rosiglitazone-treated virtual patients exhibit greater atheroma volume and more unstable plaques, and therefore higher CV risk, than pioglitazone-treated virtual patients. Early changes in circulating lipoprotein profiles during early clinical trials can be used as a biomarker to differentiate compounds that promote plaque growth and progression from those that reduce plaque growth and progression.

Analysis of hepatic gene expression from rats treated with rosiglitazone and pioglitazone using DrugMatrix®, (a molecular toxicology reference database and informatics system that contains gene expression profiles from hundreds of rat preclinical studies, Iconix Biosciences) found gene expression differences that are consistent with the observed clinical data. While the molecular target for PPARγ is not in the liver, it represents the effects of changes in metabolism of the whole animal, which impact liver lipoprotein production and clearance. These genes can be used as a biomarker to predict the changes in lipoprotein particles observed in humans and to predict CV risk to any molecule which is being used to treat the symptoms of diabetes or obesity.

The effects of defined alterations in lipoprotein particle numbers and size were simulated in the Cardiovascular PhysioLab® platform, a mathematical model of lipoprotein metabolism and plaque growth and development. Table 1 shows the changes in LDL-C and HDL-C and the shifts in LDL particles and HDL particles that were implemented based on data from a head to head comparison of rosiglitazone with pioglitazone by Deeg et al (Pioglitazone and rosiglitazone have different effects on serum lipoprotein particle concentrations and sizes in patients with type 2 diabetes and dyslipidemia. Diabetes Care. 2007 October; 30(10):2458-64.). Predictions of the effects of 24 week treatment with rosiglitazone or pioglitazone on plaque growth and stability were compared for two virtual patients representing the final lipoprotein profiles of an average patient on rosiglitazone versus an average patient on pioglitazone. All other factors (starting plaque volume, composition, inflammation, and the like) affecting plaque growth and stability were kept the same for both patients in order to isolate the effects due to lipoprotein differences resultant to the therapeutic regimen. Table 1 provides the simulated lipoprotein measures for virtual patients after 6 months of treatment.

TABLE 1
Lipoprotein Measures After 6 Months of Treatment

| Lipoprotein | Change after 6 months | Change after 6 months |
|---|---|---|
| measure | on pioglitazone (%) | on rosiglitazone (%) |
| LDL cholesterol | 11.7 | 19.6 |
| HDL cholesterol | 13.4 | 5.8 |
| Large LDL Particles | 88.4 | 71.7 |
| Small LDL Particles | −17.1 | −3.4 |
| Large HDL Particles | 16.2 | −10.5 |
| Small HDL Particles | 0.4 | −0.4 |

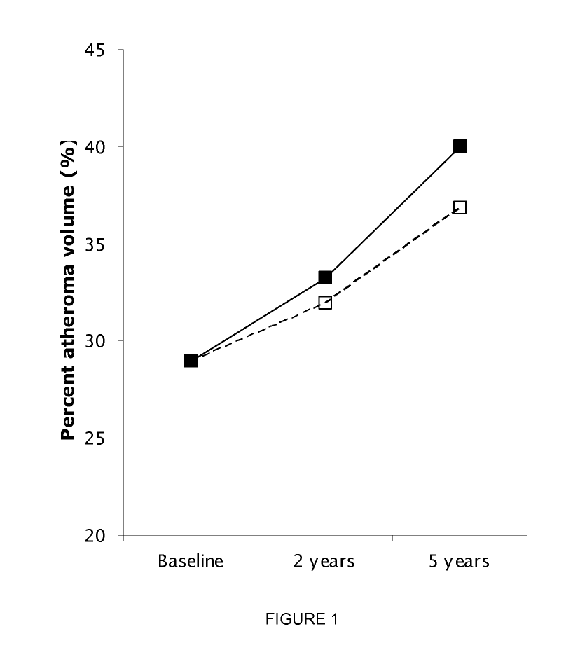

FIG. 1 provides a comparison of predicted percent atheroma volume (PAV) changes over 5 years for a virtual patient with a lipoprotein profile characteristic of treatment with rosiglitazone (filled squares) or pioglitazone (open squares). In the rosiglitazone-treated diabetic virtual patient, PAV is predicted to progress faster than in the pioglitazone-treated diabetic virtual patient.

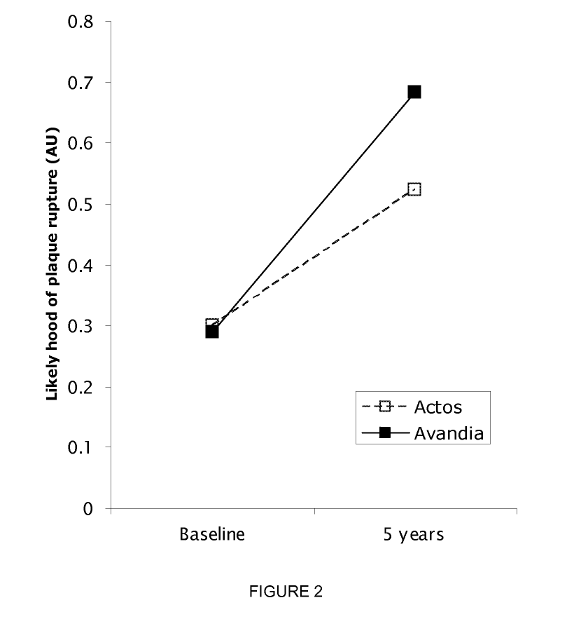

In addition to plaque volume, the effects of rosiglitazone and pioglitazone on plaque stability, i.e., the likelihood of plaque rupture due to therapy-induced changes in geometry and composition, were also predicted. FIG. 2 shows the predicted change in plaque stability after 5 years of therapy for virtual patients with profiles reflecting treatment with rosiglitazone (solid squares) or pioglitazone (open squares). The likelihood of a plaque rupture is predicted to be much greater in the virtual patient representing rosiglitazone treatment than that representing pioglitazone treatment.

In addition, analysis of hepatic gene expression from rats treated with rosiglitazone and pioglitazone using the DrugMatrix database, which contains gene expression profiles of tissues such as heart, kidney, and liver from rats treated with over 600 different compounds, revealed a panel of genes that were differentially regulated between the two drugs (Table 2). This panel of 68 probe sets were enriched in genes regulating lipid homeostasis, metabolism and transport (p-value=7e-64). These gene expression patterns are consistent with clinical data and may be useful short-term biomarkers predictive of long-term CV risk and toxicity.

TABLE 2
Relative Expression of Hepatic Genes

| | | | Average Log (10) | |
|---|---|---|---|---|
| GenBank | UniGene | | Ratio | |
| Accession No. | ID | UniGene Title | Pioglit. | Rosiglit. |
| BM390399 | Rn.9437 | 3-hydroxy-3-methylglutaryl-Coenzyme A reductase | −0.018 | 0.392 |
| NM_013134 | Rn.9437 | 3-hydroxy-3-methylglutaryl-Coenzyme A reductase | 0.086 | 0.389 |
| BG377636 | Rn.98393 | acetyl CoA transferase-like (DBSS) | 0.071 | 0.519 |
| AA899304 | Rn.4054 | acetyl-coenzyme A acetyltransferase 1 | 0.171 | 0.546 |
| BI282488 | Rn.3786 | acetyl-Coenzyme A acyltransferase 2 (mitochondrial 3- | 0.046 | 0.679 |
| | | oxoacyl-Coenzyme A thiolase) | | |
| NM_017340 | Rn.31796 | acyl-Coenzyme A oxidase 1, palmitoyl | 0.165 | 0.336 |
| NM_033352 | Rn.177278 | ATP-binding cassette, sub-family D (ALD), member 1 | 0.292 | 0.450 |
| | | (DBSS) | | |
| NM_013200 | Rn.6028 | carnitine palmitoyltransferase 1b, muscle | 0.125 | 0.984 |
| NM_012930 | Rn.11389 | carnitine palmitoyltransferase 2 | 0.030 | 0.494 |
| AF159245 | Rn.38261 | cytochrome P450, family 2, subfamily b, polypeptide 13 | −0.015 | 0.499 |
| AI454613 | Rn.91353 | Cytochrome P450, family 2, subfamily b, polypeptide 2 | 0.275 | 0.629 |
| U46118 | Rn.10489 | cytochrome P450, family 3, subfamily a, polypeptide 13 | −0.242 | 0.550 |
| M33936 | Rn.33492 | cytochrome P450, family 4, subfamily a, polypeptide 14 | 0.164 | 0.567 |
| AA893326 | Rn.33492 | cytochrome P450, family 4, subfamily a, polypeptide 14 | 0.020 | 0.328 |
| NM_031241 | Rn.23013 | cytochrome P450, family 8, subfamily b, polypeptide 1 | 0.351 | 0.394 |
| BF396857 | Rn.46942 | ELOVL family member 6, elongation of long chain fatty | −0.225 | 0.495 |
| | | acids (yeast) | | |
| U08027 | Rn.89705 | Glycerol-3-phosphate dehydrate dehydrogenase | 0.026 | 0.635 |
| | | (mtGPDH) mRNA, 3′UTR | | |
| NM_133618 | Rn.11253 | hydroxyacyl-Coenzyme A dehydrogenase/3-ketoacyl- | 0.182 | 0.409 |
| | | Coenzyme A thiolase/enoyl-Coenzyme A hydratase | | |
| | | (trifunctional protein), beta subunit | | |
| M30596 | Rn.161920 | malic enzyme 1 | −0.083 | 0.554 |
| NM_012806 | Rn.9911 | mitogen activated protein kinase 10 | 0.229 | 0.458 |
| AY081195 | Rn.40396 | monoglyceride lipase | −0.131 | 0.621 |
| BG372713 | Rn.40396 | monoglyceride lipase | −0.110 | 0.551 |
| AI713204 | Rn.40396 | monoglyceride lipase | −0.118 | 0.439 |
| M15114 | Rn.83595 | NCI_CGAP_Emb2 cDNA clone IMAGE: 4176354 | 0.052 | 0.409 |
| NM_057133 | Rn.10712 | nuclear receptor subfamily 0, group B, member 2 | 0.034 | 0.367 |
| AI385341 | Rn.9753 | peroxisome proliferator activated receptor alpha | 0.247 | 0.461 |
| AW526669 | Rn.169550 | phosphatidylinositol 3-kinase, C2 domain containing, | −0.240 | 0.692 |
| | | gamma polypeptide | | |
| NM_053551 | Rn.30070 | pyruvate dehydrogenase kinase, isoenzyme 4 | −0.148 | 0.442 |
| NM_012620 | Rn.29367 | serine (or cysteine) peptidase inhibitor, clade E, member 1 | 0.049 | 0.819 |
| D14989 | Rn.91378 | sulfotransferase family 2A, dehydroepiandrosterone | 0.112 | 0.570 |
| | | (DHEA)-preferring, member 1 | | |
| BI850137 | Rn.83595 | NCI_CGAP_Emb2 cDNA clone IMAGE: 4176354 | 0.315 | −0.414 |
| NM_012701 | Rn.87064 | adrenergic receptor, beta 1 | 0.205 | −0.177 |
| J02582 | Rn.32351 | apolipoprotein E | 0.410 | −0.324 |
| NM_031559 | Rn.2856 | carnitine palmitoyltransferase 1a, liver | 0.341 | 0.264 |
| NM_012942 | Rn.10737 | cytochrome P450, family 7, subfamily a, polypeptide 1 | 0.320 | −0.168 |
| BI292438 | Rn.79322 | elongation of very long chain fatty acids (FEN1/Elo2, | 0.334 | −0.042 |
| | | SUR4/Elo3, yeast)-like 3 (DBSS) | | |
| NM_012735 | Rn.91375 | hexokinase 2 | 0.300 | −0.330 |
| AA891362 | Rn.92789 | L-3-hydroxyacyl-Coenzyme A dehydrogenase, short | 0.375 | −0.205 |
| | | chain | | |
| BE105603 | Rn.4090 | mitogen-activated protein kinase 8 | 0.332 | −0.104 |
| BG376902 | Rn.11094 | Pyruvate carboxylase | 0.300 | −0.172 |
| AI176576 | Rn.6975 | CCAAT/enhancer binding protein (C/EBP), delta | −0.035 | −0.407 |
| NM_134382 | Rn.4243 | ELOVL family member 5, elongation of long chain fatty | −0.191 | −0.486 |
| | | acids (yeast) | | |
| NM_012565 | Rn.10447 | glucokinase | −0.220 | −1.066 |
| NM_012770 | Rn.10933 | guanylate cyclase 1, soluble, beta 2 | −0.120 | −0.465 |
| NM_012769 | Rn.87228 | guanylate cyclase 1, soluble, beta 3 | −0.120 | −0.348 |
| NM_053329 | Rn.164865 | insulin-like growth factor binding protein, acid labile | −0.263 | −0.658 |
| | | subunit | | |
| NM_017322 | Rn.9910 | mitogen-activated protein kinase 9 | −0.209 | −0.348 |
| NM_053923 | Rn.169550 | phosphatidylinositol 3-kinase, C2 domain containing, | −0.055 | −0.485 |
| | | gamma polypeptide | | |
| BI278687 | Rn.117434 | phospholipid transfer protein (DBSS) | −0.045 | −0.378 |
| NM_031976 | Rn.3619 | protein kinase, AMP-activated, beta 1 non-catalytic | −0.188 | −0.409 |
| | | subunit | | |
| NM_053994 | Rn.11126 | pyruvate dehydrogenase E1 alpha 2 | −0.171 | −0.338 |
| BM389330 | Rn.18101 | pyruvate dehydrogenase kinase, isoenzyme 3 (mapped) | −0.142 | −0.646 |
| BF407188 | Rn.15135 | RIKEN cDNA 1500016L11 (DBSS) | −0.149 | −0.448 |
| NM_017222 | Rn.85891 | solute carrier family 10, member 2 | −0.206 | −0.544 |
| J02585 | Rn.1023 | stearoyl-Coenzyme A desaturase 1 | 0.056 | −0.668 |
| AF286470 | Rn.801 | sterol regulatory element binding factor 1 | −0.118 | −0.464 |
| BF398848 | Rn.801 | sterol regulatory element binding factor 1 | −0.092 | −0.461 |
| AA945548 | Rn.91296 | transferrin | −0.222 | −0.376 |
| NM_021578 | Rn.40136 | transforming growth factor, beta 1 | −0.047 | −0.592 |
| NM_013174 | Rn.7018 | transforming growth factor, beta 3 | −0.086 | −0.380 |
| M14952 | Rn.33815 | apolipoprotein B | −0.303 | −0.255 |
| AI179334 | Rn.9486 | fatty acid synthase | −0.411 | −0.182 |
| BI288209 | Rn.44456 | glycerol-3-phosphate acyltransferase, mitochondrial | −0.319 | −0.183 |
| U36771 | Rn.44456 | glycerol-3-phosphate acyltransferase, mitochondrial | −0.239 | −0.156 |
| BI281656 | Rn.39132 | guanine nucleotide binding protein, alpha stimulating, | −0.386 | 0.107 |
| | | olfactory type | | |
| AI406700 | Rn.9737 | perilipin | −0.187 | 0.137 |
| NM_022627 | Rn.15423 | protein kinase, AMP-activated, beta 2 non-catalytic | −0.432 | −0.211 |
| | | subunit | | |
| NM_031131 | Rn.24539 | transforming growth factor, beta 2 | −0.378 | 0.020 |

Biomarkers are useful for understanding the systemic complexities of a disease that are not readily measurable. The selection and interpretation of biomarkers is dependent on the relationship between the biomarker and the quantity of interest. In addition, a biomarker's predictive value depends on the conditions (experimental protocol, measurement time) under which it is measured. The present invention provides biomarkers comprising as few as 4 genes that are useful for determining assessing the pro- or anti-atherosclerotic effect of a diabetes therapy. These biomarkers (and the genes from which they are composed) may also be used in the design of improved diagnostic devices.

The biomarkers of the invention comprise a measurement of expression of each of a plurality of genes selected from those listed in Table 2. Preferably the plurality of genes comprises at least three, at least five or at least eight genes selected from Table 2. Preferably, the plurality of genes includes at least one of malic enzyme 1 (accession No. M30596), perilipin (Accession No. AI406700), pyruvate carboxylase (Accession No. BG376902), acetyl-Coenzyme A acyltransferase 2 (mitochondrial 3-oxoacyl-Coenzyme Athiolase))Accession No. BI282488), 3-hydroxy-3-methylglutaryl-Coenzyme A reductase (accession No. BM390399), and apolipoprotein E (Accession No. J02582).

“Biomarker” as used herein, refers to a combination of variables, weighting factors, and other constants that provides a unique value or function capable of answering a classification question. A biomarker may include as few as one variable. Biomarkers include but are not limited to linear equations comprising sums of the product of gene expression log ratios by weighting factors and a bias term.

“Variable” as used herein, refers to any value that may vary. For example, variables may represent relative or absolute amounts of biological molecules, such as mRNA or proteins, or other biological metabolites. Variables may also represent dosing amounts of test compounds.

Diagnostic reagent sets may include reagents representing a subset of genes found in the set of 68 consisting of less than 50%, 40%, 30%, 20%, 10%, or even less than 5% of the total genes. In one preferred embodiment, the diagnostic reagent set is a plurality of polynucleotides or polypeptides representing specific genes in a sufficient or necessary set of the invention. Such biopolymer reagent sets are immediately applicable in any of the diagnostic assay methods (and the associate kits) well known for polynucleotides and polypeptides (e.g., DNA arrays, RT-PCR, immunoassays or other receptor based assays for polypeptides or proteins).

As described above, the methodology described here is not limited to polynucleotide data. The invention may be applied to other types of datasets. For example, proteomics assay techniques, where protein levels are measured or protein interaction techniques such as yeast 2-hybrid or mass spectrometry also result in large dataset, which could be utilized to infer the relative expression of polypeptides represented in the biomarkers of the present invention.

The diagnostic reagent sets of the invention may be provided in kits, wherein the kits may or may not comprise additional reagents or components necessary for the particular diagnostic application in which the reagent set is to be employed. Thus, for polynucleotide array applications, the diagnostic reagent sets may be provided in a kit which further comprises one or more of the additional requisite reagents for amplifying and/or labeling a microarray probe or target (e.g., polymerases, labeled nucleotides, and the like).

A variety of array formats (for either polynucleotides and/or polypeptides) are well-known in the art and may be used with the methods and subsets produced by the present invention. In one preferred embodiment, photolithographic or micromirror methods may be used to spatially direct light-induced chemical modifications of spacer units or functional groups resulting in attachment at specific localized regions on the surface of the substrate. Light-directed methods of controlling reactivity and immobilizing chemical compounds on solid substrates are well-known in the art and described in U.S. Pat. Nos. 4,562,157, 5,143,854, 5,556,961, 5,968,740, and 6,153,744, and PCT publication WO 99/42813, each of which is hereby incorporated by reference herein.

Alternatively, a plurality of molecules may be attached to a single substrate by precise deposition of chemical reagents. For example, methods for achieving high spatial resolution in depositing small volumes of a liquid reagent on a solid substrate are disclosed in U.S. Pat. Nos. 5,474,796 and 5,807,522, both of which are hereby incorporated by reference herein.

### EXAMPLES

The following examples are provided as a guide for a practitioner of ordinary skill in the art. The examples should not be construed as limiting the invention, as the examples merely provide specific methodology useful in understanding and practicing an embodiment of the invention.

### Example 1

### Development of Expression Profile

Male Sprague-Dawley (Crl:CD® (SD)(IGS)BR) rats (Charles River Laboratories, Portage, Mich.), weight matched, 7 to 8 weeks of age, were housed individually in hanging, stainless steel, wire-bottom cages in a temperature (66-77° F.), light (12-hour dark/light cycle) and humidity (30-70%) controlled room. Water and rodent diet were available ad libitum throughout the 5 day acclimatization period and during the 5 day treatment period. Housing and treatment of the animals were in accordance with regulations outlined in the USDA Animal Welfare Act (9 CFR Parts 1, 2 and 3).

Rats (three per group) were dosed daily at either a low or high dose. The low dose was an efficacious dose estimated from the literature and the high dose was an empirically-determined maximum tolerated dose, defined as the dose that causes a 50% decrease in body weight gain relative to controls during the course of the 5 day range finding study. Animals were necropsied on days 0.25, 1, 3, and 5. Up to 13 tissues (e.g., liver, kidney, heart, bone marrow, blood, spleen, brain, intestine, glandular and nonglandular stomach, lung, muscle, and gonads) were collected for histopathological evaluation and microarray expression profiling on the Affymetrix Rat Whole Genome RG230 v2 platform. In addition, a clinical pathology panel consisting of 37 clinical chemistry and hematology parameters was generated from blood samples collected on days 3 and 5.

Gene expression profiling, data processing and quality control were performed using protocols recommended by. Briefly, liver samples from 3 rats were chosen at random from each treatment and control group for each timepoint for expression profile analysis on the Affymetrix Rat Whole Genome RG230 v2 microarray (Affymetrix, Santa Clara, Calif.). Log transformed signal data for all probes were array-wise normalized using the Affymetrix MASS algorithm. Expression log ratios of base 10 (log(10) ratios) were computed as the difference between the logs of the averaged normalized experimental signals and the averaged normalized time-matched vehicle control signals for each gene.

TABLE 3
shows which experiments were analyzed.

| | Dose | | | |
|---|---|---|---|---|
| | (mg/kg/ | Time | | Route of |
| Compound | d) | (days) | Vehicle | Administration |
| PIOGLITAZONE | 1500 | 3 | CORN OIL | ORAL GAVAGE |
| PIOGLITAZONE | 1500 | 5 | CORN OIL | ORAL GAVAGE |
| PIOGLITAZONE | 300 | 3 | CORN OIL | ORAL GAVAGE |
| ROSIGLITAZONE | 1800 | 3 | CORN OIL | ORAL GAVAGE |
| ROSIGLITAZONE | 1800 | 5 | CORN OIL | ORAL GAVAGE |

A series of oligonucleotide probes taken from each gene was selected using the following criteria: (1) gene probes that rosiglitazone induced at least two-fold in both experiments but that pioglitazone induced less than two-fold, caused no change, or repressed in at least two of three experiments; (2) gene probes that rosiglitazone repressed by at least two-fold in both experiments but that pioglitazone repressed less than two-fold, induced, or caused no change in at least two of three experiments; (3) gene probes that pioglitazone induced at least two-fold in two of three experiments but that rosiglitazone induced less than two-fold, caused no change, or repressed in both experiments; (4) gene probes that pioglitazone repressed by at least two-fold in at least two of three experiments but that rosiglitazone repressed less than two-fold, induced, or caused no change in at least two of three experiments.

Various modifications and variations of the described biomarkers and methods of the invention will be apparent to those of skill in the art without departing from the scope and spirit of the invention. Although the invention has been described in connection with specific preferred embodiments, it should be understood that the invention as claimed should not be unduly limited so such specific embodiments. Indeed, various modifications of the described modes for carrying out the invention that are obvious to those skilled in the art are intended to be within the scope of the following claims.

We claim:

1. A biomarker for assessing atherosclerotic potential of an anti-diabetic therapy in a subject, said biomarker comprising a measurement of expression of each of a plurality of genes selected from those listed in Table 2.

2. The biomarker of claim 1, wherein the plurality of genes comprises at least three, at least five or at least eight genes selected from Table 2.

3. The biomarker of claim 1, wherein the plurality of genes includes at least one of malic enzyme 1 (accession No. M30596), perilipin (Accession No. AI406700), pyruvate carboxylase (Accession No. BG376902), acetyl-Coenzyme A acyltransferase 2 (mitochondrial 3-oxoacyl-Coenzyme Athiolase) Accession No. BI282488), 3-hydroxy-3-methylglutaryl-Coenzyme A reductase (accession No. BM390399), and apolipoprotein E (Accession No. J02582).

4. A method for testing whether a compound will induce atherosclerosis in a test subject, the method comprising:
administering a dose of the compound to at least one test subject;
after a selected time period, obtaining a biological sample from the at least one test subject;
measuring the expression levels in the biological sample of at least a plurality of genes selected from those listed in Table 4;
determining whether the sample is in the positive class for induction of atherosclerosis using a classifier comprising at least the plurality of genes for which the expression levels are measured.

5. The method of claim 4, wherein the biological sample comprises liver tissue.

6. The method of claim 4, wherein the dose administered does not cause histological or clinical evidence of atherosclerosis at about 7 days, about 14 days, or about 21 days.

7. The method of claim 4, wherein the expression levels are measured as log₁₀ ratios of compound-treated biological sample to a compound-untreated biological sample.

8. The method of claim 4, wherein the classifier is a linear classifier.

9. The method of claim 4, wherein the classifier is a non-linear classifier.

10. The method of claim 4, wherein the selected period of time is about 7 days or fewer.

11. A reagent set comprising a plurality of polynucleotides or polypeptides representing a plurality of genes selected from those listed in Table 4.

12. The reagent set of claim 11, comprising a plurality of genes includes at least 4 genes selected from those listed in Table 4, the 4 genes having at least 2% of the total impact of all of the genes in Table 4.

13. The reagent set of claim 11, comprising a plurality of genes includes at least 8 genes selected from those listed in Table 4, the 8 genes having at least 4% of the total impact of all of the genes in Table 4.

14. The reagent set of claim 11, wherein the reagent set is based on subsets of genes randomly selected from Table 4, wherein the subset includes at least 4 genes having at least 1, 2, 4, 8, 16, 32, or 64% of the total impact.

15. The reagent set of claim 11, wherein the plurality of genes consists of fewer than 1000 polynucleotides or polypeptides.

16. The reagent set of claim 15, wherein the plurality of genes consists of fewer than 200 polynucleotides or polypeptides.

17. The reagent set of claim 15, wherein the plurality of genes consists of fewer than 8 polynucleotides or polypeptides.

18. The reagent set of claim 11, wherein the reagent set consists essentially of polynucleotides or polypeptides selected from Table 4.

19. An apparatus for predicting whether a compound will induce atherosclerosis in a test subject comprising a reagent set of claim 11.
